(19) United States
(12) Patent Application Publication
(10) Pub. No.: US 2010/0317966 A1
(43) Pub. Date: Dec. 16, 2010

(54) Hybrid-multimodal magneto-optical contrast markers

(75) Inventors: Nolwenn Le Bouch, Quebec (CA); Ozzy Mermut, Quebec (CA)

Correspondence Address: MERCHANT & GOULD PC, P.O. BOX 2903, MINNEAPOLIS, MN, 55402-0903, US

(73) Assignee: Institut National D'Optique, Quebec (CA)

(21) Appl. No.: 12/802,349

(22) Filed: Jun. 4, 2010

Related U.S. Application Data
(60) Provisional application No. 61/184,520, filed on Jun. 5, 2009.

(51) Int. Cl. A61B 5/05; C07D 209/20; C07D 487/22; C01B 13/00; C01B 21/20

(52) U.S. Cl. 600/426; 548/494; 540/145; 423/579; 423/387

(57) ABSTRACT

A contrast marker for use in imaging applications, wherein the marker is sensitive to an applied magnetic field through the formation of radical pair species and wherein the marker is independently magnetically sensitive, optically sensitive, and magneto-optically sensitive.

This application claims benefit of U.S. Ser. No. 61/184,520, filed Jun. 5, 2009 and which application is incorporated herein by reference. To the extent appropriate, a claim of priority is made to the above disclosed application.

### FIELD OF THE INVENTION

The present invention is directed to a concept/scheme of a contrast agent to be used for imaging with the concept of magneto-optic imaging platforms that are novel forms of fused modality for imaging from optical signals affected by an external magnetic field. The contrast agent of the present invention has radical pairs with parallel electrons spins and needs to be optical sensitive, magneto sensitive and magneto-optic sensitive.

### BACKGROUND OF THE INVENTION

A medical contrast compound is a biocompatible chemical substance used to enhance the contrast of structures or fluids within the body in medical imaging. For example, iodine and barium sulfate are used as agents in X-ray applications; paramagnetic species and super paramagnetic species are used in magnetic resonance imaging (MRI) applications, and phospholipid microspheres and microparticles of galactose are used in ultrasound applications.

More recent advances in imaging applications for diagnostic and treatment purposes have been made particularly in the field of optical imaging, using electromagnetic radiation in the visible and near-infrared (NIR) part of the spectrum. Indeed, optical imaging techniques, and photodynamic therapy are relatively recent tools, and permit more specific imaging, as well as better targeted drug delivery. Optical imaging often makes use of luminescent contrast agents (producing either fluorescence or phosphorescence) that can target specific biomolecules or physiological processes within a living organism. However, living tissues induce scattering of light at the visible and NIR wavelengths, limiting optical imaging spatial resolution. Coupling optical imaging with a structural imaging modality is thus currently a very active field of research.

In this context, U.S. Pat. No. 7,519,411 (Long), assigned to the same Assignee as the instant application teach using magneto-optical effects to optically probe or monitor a biochemical/physiological process in vivo in the case of photodynamic therapy using a simple system combining a highly sensitive optical device using weak magnetic fields. The present invention is based on the same fundamental physics arising from the perturbation of optical electronic states by a magnetic field as described in Long. Briefly, a magneto-optic effect (MOE) refers to a perturbation of an optical emission imparted by application of a magnetic field. As illustrated in FIG. 4 an external magnetic field can alter the reaction rate and/or product distribution in reactions involving radical pairs. The orientation of the electron spins of photoexcited species is important in determining their magnetic susceptibility. The spin exchange in a radical pair system, and hence the kinetics and yield of luminescence, are mainly governed by hyperfine coupling of the unpaired electrons with the magnetic moments of the nuclei and the interaction of these electrons with external magnetic fields. Specifically, the underlying physics relies on Zeeman splitting and hyperfine coupling (hfc) effects which produce changes in the electronic states, and hence are detectable in the optical domain by transitions related to excited electronic states (fluorescence, delayed fluorescence, phosphorescence, luminescence, bioluminescence, chemiluminescence etc). The fundamental requirement for achieving contrast is that of parallel electron spins (i.e. molecular species in the triplet state), which renders a material magnetically susceptible (FIG. 1, identified as Prior Art).

The potential of the magneto-optic technique as described by Long for photodynamic therapy (PDT) can be opened to other medical treatment applications. Molecular Imaging is probably the major field in which this new technology can be developed. Molecular imaging maps the location of specific molecules and biomaterials within living tissue. As such, it has the potential to diagnose a disease, to monitor the course of that disease, and possibly facilitate treatment. Molecular imaging has grown into a promising new strategy for the diagnosis, evaluation and treatment of disease. Interest in use of the technology has exploded in recent years, thanks to advances in cell biology, biochemical agents, and computer analysis. Medical imaging industries, as well as those serving or investing in medical imaging companies, will want to keep abreast of this new market. Furthermore each medical imaging modality has unique strengths and limitations and it is often through the use of multiple modalities that a complete assessment of a patient is achieved. The prior art addresses molecular contrast agent schemes for multimodality approaches in the form of combining two techniques so that the advantages of each reduce the disadvantages of the other mutually.

For example, Nielsen (PCT/IB2006/052543 entitled “Optical Imaging” describes a probe molecule which contains a donor (D) and an acceptor (A) entity with the following properties: if the donor is optically excited, an electron is transferred to the acceptor resulting in a charge-transfer complex in a singlet state. This complex can cross over to a triplet state through intersystem crossing with a rate dependent on the strength and/or the direction of an external magnetic field. Both states, the triplet and the singlet state, may have separate decay channels by which the excitation energy is transferred to the environment. For instance, the singlet state can predominantly emit fluorescence radiation, and the triplet state can predominantly emit phosphorescence radiation. Nielsen uses the concept of magnetic field effect, but emphasizes the use of weak fields. Nielsen teaches that the molecule is designed for use in a spatially inhomogeneous magnetic field in order to preselect photons from a specific location and thus improve optical imaging spatial resolution by rejecting scattered photons; in essence, the compound enables spatial selectivity for optical imaging, allowing improved structural information.

It follows from the teachings of Nielsen that Nielsen is mostly concerned with the following features:
the probe is attachable to an object of interest,
the probe comprises a donor and an acceptor,
a linker molecule couples the donor to the acceptor
a charge-transfer takes place when the donor is optically excited
additional molecule can be bound to the probe
one field of application of the invention is tumour detection

The molecular probe concept described herein retains some of the same basic features described above, like the donor-acceptor complex and the need of charge-transfer to yield radical states that are subsequently sensitive to magnetic perturbed luminescence; it however extends the required properties toward a specific goal of observing radical species phenomena in physiology through a unique hybridized synergistic magneto-optical approach. While the probe described herein comprises also a donor and an acceptor, both moieties do not need to be coupled, because the formation of radical pairs and charge-transfer can happen either inside the structure of the contrast marker, or either in an interaction with a surrounding endogenous substrate. The contrast marker is sensitive to the magnetic field through the formation of radical pair species that are paramagnetic in the triplet state. The radical-pair diffusion needs to be limited for enhanced recombination and longer lifetimes. Our probe will present an increase fluorescence when B field apply and eventually our probe is not limited to detect tumors because the radical pairs susceptible to the Magneto-Optic Effect are not only related to singlet oxygen or reactive oxygen species produced in various diseases but also in other bioprocesses (inflammation, ketamine response, enzymatic reactions, etc.) as long as radicals are produced and could be monitored.

Also of interest is PCT/US2008/067009, WO 2009/045579 A2 entitled “Multimodal Imaging Probes for In Vivo Targeted and Non-targeted Imaging and Therapeutics”. This reference relates to the combination of different chemicals to form a multimodal probe that will be sensitive to several types of imaging techniques like magnetic resonance imaging (MRI), positron emission tomography (PET), near-infrared imaging (NIR), electron spin resonance (ESR). Nanoparticle-based imaging probes are provided comprising a nanoparticle attached to one or more imaging materials. Suitable contrast agents include, but are not limited to MRI materials, ESR materials, NIR materials, PET materials, and the like. The nanoparticle can itself be a moiety that provides a detectable signal (e.g., a quantum dot) in which case the nanoparticle/agent combination can provide at least two different detection modalities. For example in the case of magnetic field and optical sensibility, they combine an MRI agent (chelated Gd³⁺) with a nanoparticle-based probe for NIR optical detection. The combination of detection modes with the help of a multifunctional contrast agent as described in the above-cited patent is fundamentally different from a coupling of detection where one mode of detection implies a change in response of another mode of detection as it appears in our concept.

PCT/US2004/026479, entitled is “Imaging Pathology” discusses bifunctional magnetic resonance/optical probes. The probe is used to differentiate a diseased tissue from normal host tissue. This application also relates to the use of a bifunctional probe including an optical imaging moiety and a magnetic resonance imaging moiety in the manufacture of a medicament to differentiate a diseased tissue from a normal host tissue in a subject under intra-operative conditions to remove or explore the diseased tissue. In this patent the two modes of diagnostic to differentiate a normal tissue to a diseased one are also optical and magnetic but this scheme does not use the magneto-optic detection. The probe is built on the same model as the patent PCT/US2008/067009, WO 2009/045579 A2, they use two different moieties that respond respectively to two different detection modes to form the contrast agent. This mode of detection does not use the synergistic combination of one detection having an input on the other.

The main drawbacks of these patents are that they present a straightforward scheme of linking two independent reporters that do not necessarily interact to carry supplemental physiological information than what could be obtained if used separately. They merely allow circumventing instrumental method limitations of a particular modality. This means that one type of probe (optical, magnetic, etc.) is associated with one specific response of the system. They, of course, can be chemically combined (e.g., through covalent attachment) to build a new material but the response will remain two parallel detection channels.

There exist numerous other patents that present contrast agents that permit a combination of technologies to work but their scheme of detection are always parallel detection. Compared to these examples, our contrast agent and the technology related are innovative; they do not represent a combination of already known technologies but a new postulate and works in a complementary way of technologies, one technology having an input on the other. What we propose here is the concept of an hybridized multimodal contrast agent that is two-field sensitive (optical and magnetic) that, in addition of operating only independently in each field, also operates synergistically between the magnetic and optical fields offering the capability to monitor and observe physiological processes (related to the radical pair mechanism) at the molecular level.

The unique nature of these magneto-optic agents is that, according to its design, they can act both as contrast markers and as therapeutic drug in one platform.

### SUMMARY OF THE INVENTION

The present invention thus concerns a contrast marker scheme which overcomes the shortcomings of the prior art.

The probe must provide the two essentials properties of the magneto-optic concept, i.e. the contrast marker is magneto-optic sensitive (through the formation of parallel electron spins) and fulfills the multimodal operation scheme (FIG. 3), which means it needs to be independently magnetic sensitive, optical sensitive and magneto-optic sensitive.

The contrast marker scheme according to a preferred embodiment of the invention is sensitive to the magnetic field through the formation of radical pair species.

In accordance with one aspect of the invention, there is provided a contrast marker for use in imaging applications, wherein the marker is sensitive to an applied magnetic field through the formation of radical pair species and wherein the marker is independently magnetically sensitive, optically sensitive, and magneto-optically sensitive.

In one embodiment, the marker may take the form of a Donor-Acceptor (D-A) complex. When the donor is optically excited, an electron is transferred to the acceptor resulting in a charge transfer complex in a singlet state. This complex can cross over to a triplet state with a rate depending on the strength or the direction (or both) of an external magnetic field. The concept of Donor-Acceptor is known, but in the present invention, the D-A complex is combined with the magnetic-field effect as defined in FIG. 5 to provide a unique type of physiological information related to radicals.

### BRIEF DESCRIPTION OF THE DRAWINGS

FIG. 1: Fundamental chemical physics required for MO contrast. Radical recombination leading to: A) Stable molecule—MO insensitive vs. B) magnetically unstable molecules—MO sensitive.

FIG. 2. Proposed application for the contrast agent of the present invention: A) direct interaction of the contrast agent (MOMAT) with target molecule to generate radical ion pairs (RIP) which are subsequently susceptible to a MO effect. B) interaction of a RIP contrast molecules with byproducts or regulatory molecules, which affect the MO response of the contrast marker. Note: D-A=donor-acceptor molecule

FIG. 3: Multimodal operation schemes: (a) optical only (i.e. fluo/phospho emission), (b) magnetic only (i.e. MRI or weak β-field), (c) magneto-coupled optic.

FIG. 4: The origin of magneto-optic effects, MOE, in the contrast compound concept scheme arise from: (A) the Zeeman splitting of degenerate states, T₀, T₊₁, T₋₁, in response to increasing B-field; and (B) the hyperfine coupling, hfc, between donor-acceptor (D-A) singlet and triplet states.

FIG. 5: The magneto-optic effect on an Acceptor-Donor complex

FIG. 6: Commercial model of a magneto-optic photosensitizer: Cu(II) phtalocyanine photosensitizer 10.

FIG. 7: Custom model of a magneto-optic photosensitizer: Fulleren-Porphyrin

FIG. 8: Custom model of a magneto-optic photosensitizer: CdSe-Porphyrin

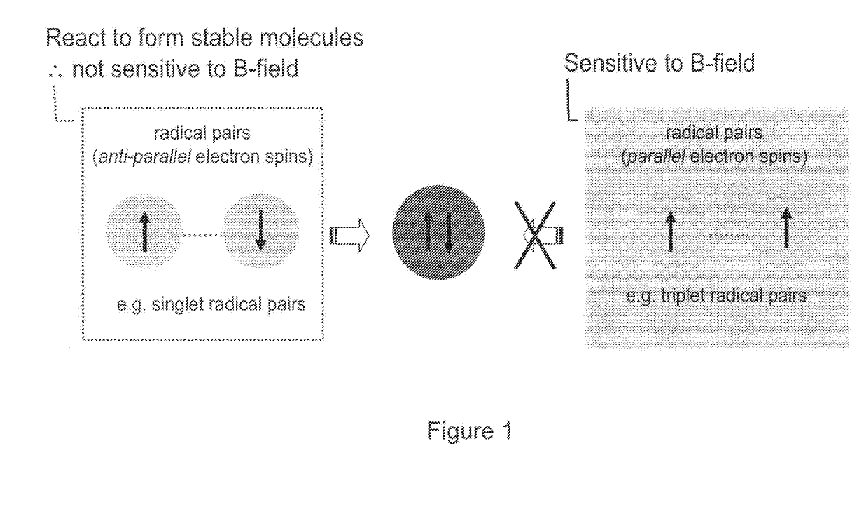

### DESCRIPTION OF A PREFERRED EMBODIMENT OF THE INVENTION

The present invention concerns a contrast agent that is two-field sensitive (optical and magnetic) and in addition to operating only independently in each field, also operates synergistically between the magnetic and optical fields.

The present invention introduces a novel form of fused modality for molecular imaging, more specifically, for imaging under different platforms. Using this novel hybridized modality, the new magneto-optic platforms serve as imaging tools for early-stage detection and contrast of biomolecular constituents aimed at identifying and discriminating healthy vs. diseased states in vivo.

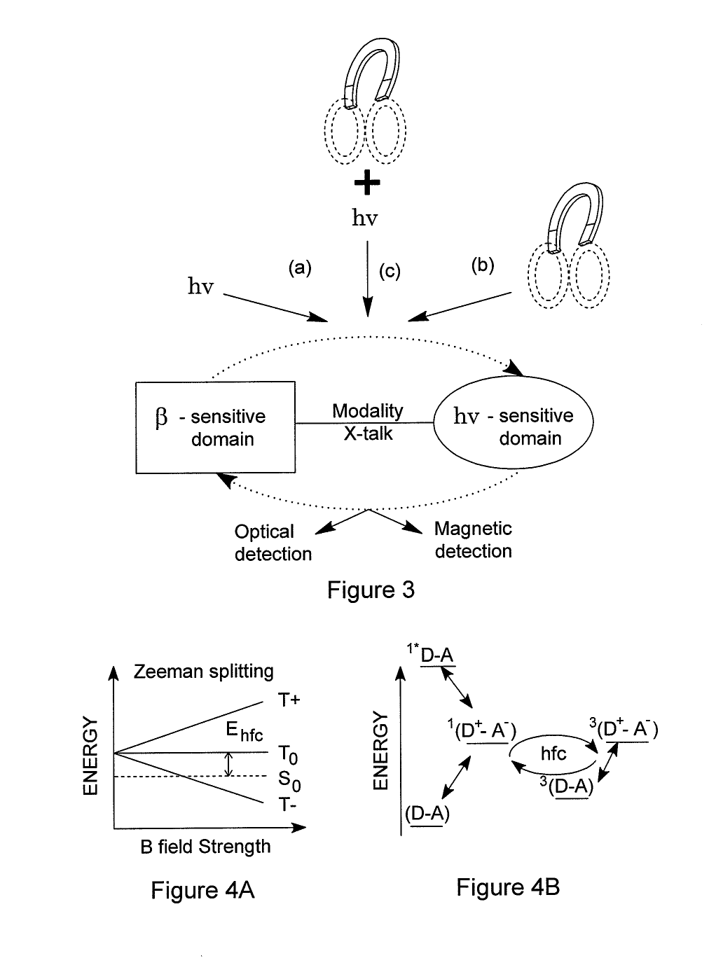

The probe must provide the two essential properties of the magneto-optic concept, i.e. the contrast marker needs to be magneto-optic sensitive (through the formation of parallel electron spins) and it needs to fulfill the multimodal operation scheme (FIG. 3), which means it needs to be independently magnetic sensitive, optical sensitive and magneto-optic sensitive.

The way the material concept is designed corresponds to this multimodality. The present invention uses the benefits of two already established biomedical techniques: magnetic field (MRI, for example, but also weak field options) and optics, but additionally provides a third opportunity for imaging which is the concept of magneto-optic response, meaning the optical response will depend on the magnetic field applied. The multimodal imaging probes or platforms existing usually refer to a combination of existing techniques rather than a fused modality. The novelty and uniqueness of the present invention relays of the hybridization of the multimodal concepts which are first of all two-field sensitive (optical and magnetic) and in addition of operating independently in each field, also operates synergistically between the magnetic and optical fields (FIG. 3). This additional “fused” concept offers the capability to monitor molecular-level processes in vivo derived from a third unexploited modality. In short, one obtains 3 modes of contrast for the price of 2.

Long's patent focused on PDT treatment using weak fields. The present invention combines the PDT treatment potential with wider possibilities such as: monitoring oxygen concentration, studying particular disease pathways at the molecular level, observing enzymatic processes, notably those related to reactive oxygen species (e.g. cell signaling, inflammatory response, aging processes, etc.). Moreover, the principles of the present invention are wide enough to be used to monitor many biomolecular and physiological processes that are based on chemical reaction pathways producing radical pair intermediaries. Examples of the importance of the radical pair mechanism in biology and medicine include many enzymatic reactions, diseases action and even therapies such as photodynamic therapy in cancer treatment. Potentially, manipulating these bioprocesses through adjustment or modulation of the applied magnetic field(s), can provide control over a biochemical outcome while concurrently monitoring the perturbation optically. The use of stronger magnetic fields is also contemplated in order to take advantage of the omnipresence of MRI machines in hospitals.

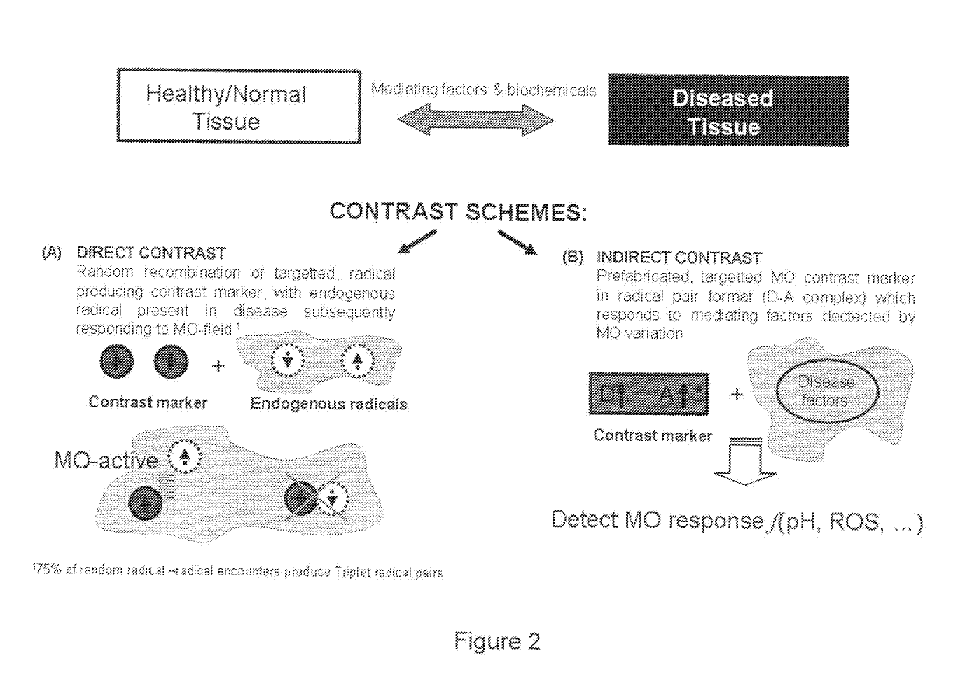

The present invention thus concerns a contrast agent aimed for contrast imaging in early detection/identification of disease and disease pathway (by distinguishing healthy vs. diseased tissues) through biomolecules, biosignaling events, biomechanisms, variations in biopathways, or a combination thereof (FIG. 2).

The contrast agent of the present invention does not have the goal to be related to “treatment” of the tissue/region of interest like in PDT. With the present invention, there is no intention to cause this photocytotoxicity or to be used only as PDT agent. Reactive oxygen species are only involved to the extent of identifying them for early detection of disease.

The contrast marker is not a photosensitizer (i.e., not a PDT agent), it only needs to be sensitive to the magneto-optic concept to be used in platforms using this same concept.

As shown in FIG. 2, the contrast marker mechanism, that is to say the formation of radical pairs and charge-transfer, can happen either inside the structure of the contrast marker, or either in an interaction with a surrounding endogenous substrate.

As the contrast marker of the present invention is not restricted to PDT applications, the radical pairs susceptible to the Magneto-Optic Effect need not be related to singlet oxygen or reactive oxygen species produced by either pathways in PDT. In fact, there is no PDT pathway required. The relevant radical pairs monitored are determined by the precise bioprocess examined (inflammation, ketamine response, enzymatic reactions) i.e. could be nitroxyl radicals, radical pairs associated with tryptophan & FADH, radical generated from donor-acceptor porphyrin complexes, radicals generated from enzymatic processes, etc.

The contrast marker according to a preferred embodiment of the invention is sensitive to the magnetic field through the formation of radical pair species that are paramagnetic in the triplet state (parallel electron spins).

The probe must provide the two essential properties of the magneto-optic concept, i.e. the contrast marker needs to be magneto-optic sensitive (through the formation of parallel electron spins) and it needs to fulfill the multimodal operation scheme (FIG. 3), which means it needs to be independently magnetic sensitive, optical sensitive and magneto-optic sensitive.

The contrast marker that can be magnetically or optically activated will reemit luminescence and this luminescence signal response can be measured through different optical parameters like luminescence intensity, lifetime, spectral properties, etc.

On the other side the contrast marker response, optically and/or magnetically-activated, can also be measured in magnetic response through the measure of the resonance spectral response induced by a radiofequency pulse.

In general the probe may take the form of a Donor-Acceptor complex as this one way to achieving the magneto-optic effect. When the donor is optically excited, an electron is transferred to the acceptor resulting in a charge transfer complex in a singlet state. This complex can cross over to a triplet state with a rate depending on the strength or the direction (or both) of an external magnetic field. Numerous molecules, organic or inorganic can be considered as donor and acceptor molecules.

The acceptor by definition needs to be able to accept electrons. Some examples could be but are not restricted to: fullerenes, quantum dots (CdSe, CdTe, ZnS, etc.), carbon nanotubes, substrate, etc.

The donor can be, but are not restricted to: porphyrins (with varying metal centers), phtalocyanines, quantum dots, etc.

Both entities can be covalently linked but they also can only be in close contact. The charge-transfer necessary for the complex formation is eased with a short distance between the donor and the acceptor. However, an endogenous substrate can serve as an acceptor in the complex if the donor-acceptor distance is short enough to allow charge-transfer.

Any other chemical component could be added to the generic probe as desired to increase its multimodality. That means that an ESR, EPR, MRI, NIR material for example, could be covalently attached to the initial contrast marker to make it also ESR, EPR, MRI, or NIR active. This will be in addition to the fundamental property of the magneto-optical sensitivity.

Besides chemical modifications, tuning the encapsulation or delivery system for the compound can also be an option to target the detection site and maximize the change in luminescence response to magnetic field. There are different options for the generic term encapsulation or delivery system. For example, nanosized materials have unique properties that have made them useful for delivering drugs. Classes of nanosized systems used in the pharmaceutical industry are liposomes, nanocrystals, micelles, colloidal particles, quantum dots, and dendrimers, to name but a few. Liposomal confinement can lengthen the luminescence lifetime of the PS and enhance magnetic field effects (MFEs). Having important applications in nanomedicine, liposome technology was developed to improve the pharmocokinetics and the bioavailability of therapeutics. The incorporation of the magneto-optically active molecule complexes in liposomes has previously been shown to enhance their phototoxicity significantly. Micelles provide an interesting situation where both polar and nonpolar phases are present in dynamic equilibrium. Due to their dipolar or multipolar character, D, A pairs prefer to stay at the interfacial regions. In micelles, emissions from D, A pairs joined by flexible links increase considerably. Dendrimers, due to their interior void space and surface functional groups are also well-suited for use as carrier molecules in drug delivery. Dendritic encapsulation of functional molecules allows for the isolation of the active site, a structure that mimics the structure of active sites in biomaterials because dendritic scaffolds separate internal and external functions.

### EXAMPLES

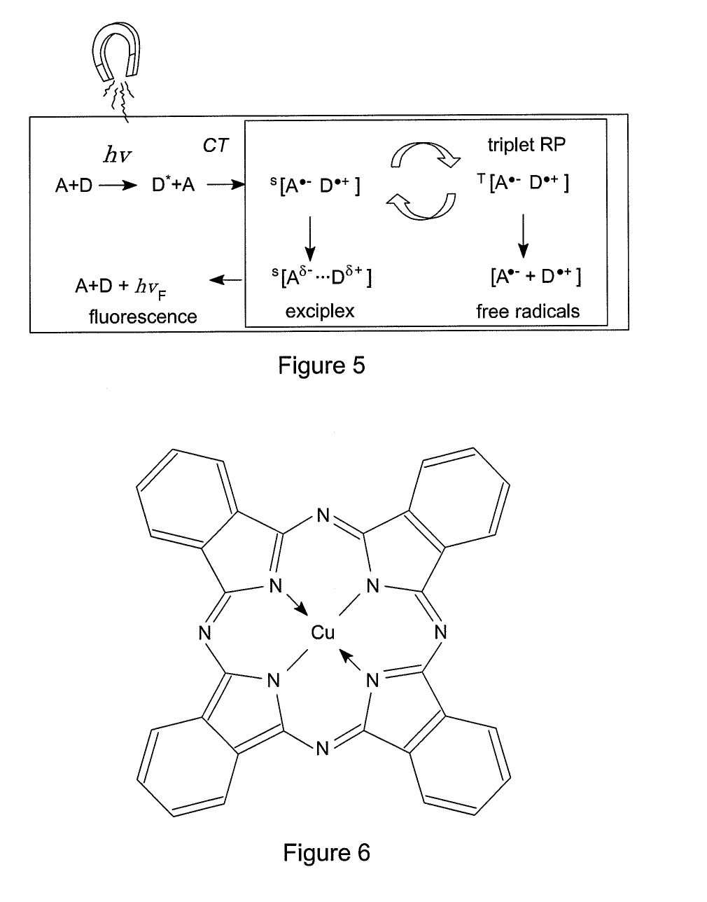

In the following, exemplary embodiments of the probe will be described. These embodiments do not limit the use of other chemical compounds, organic or inorganic to form a probe. A Donor-Acceptor complex is a molecular system composed of electron deficient (A) and electron rich (D) moieties that upon photoexcitation undergo Charge-Transfer (CT) to yield radical states that are subsequently sensitive to magnetically-perturbed luminescence (FIG. 5). The D and A entities may either be deliberately coupled through a chemical bond, or the photosensitizer may interact with a substrate or other endogenous molecules present in the environment which serve as a D or A.

The following examples are offered to illustrate, but do not limit the claimed invention.

### 1. First Example: Phtalocvanines

Commercial phtalocyanines derivatives (FIG. 6) are well known PDT agents and good examples of the possible interaction with an endogeneous substrate when forming the D-A complex under the magneto-optic effect. Phtalocyanines can undergo photo-initiated electron transfer, or hydrogen abstraction to generating transient radical species, after absorbing visible light; these effects are relevant to photosensitization in the absence of oxygen or in more polar environments. The electrons spins of photoexcited species can be utilized to affect the photophysical processes involved in photosensitization. Both luminescence intensity and lifetime change upon exposure of the photoexcited molecule to weak magnetic field.

From this family of commercial derivatives of phtalocyanines, porphyrin and chlorin could also be taken as examples for MOE.

### 2. Second Example

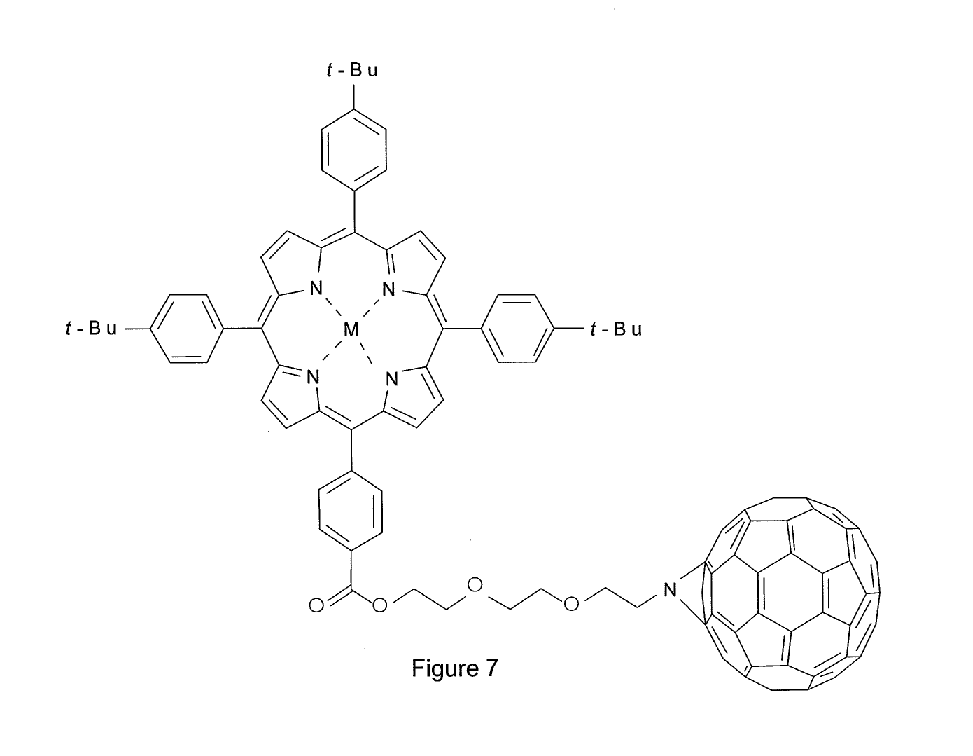

The first custom magneto-optic photosensitizer conceptualized was a model D-A designed by bridging a standard and commercial PS, porphyrin (with varying metal centers), with a fullerene (C₆₀) molecule (FIG. 7). Fullerenes are ideal acceptors (borrowed from model photosynthetic systems), capable of reversibly accepting multiple electrons in solution and generation of long-lived and stable radical states. They accelerate charge separation and slow down charge recombination making them ideal for magneto-optic photosensitization.

In this family of compounds, the porphyrin moiety can be customized by modifying the metal centers (Cu, Zn, . . . ). Other organic compounds from the family of the fullerene could also be used (carbon nanotubes, C₇₀, . . . ) as acceptor moieties.

### 3. Third Example

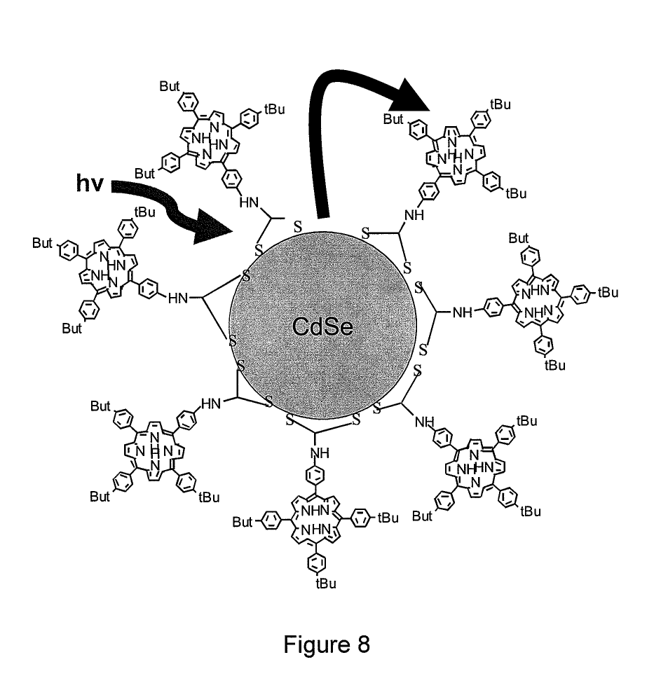

A second example of a custom magneto-optic photosensitizer was fabricated from quantum dots (QDs) of CdSe and CdTe linked to a porphyrin moiety (FIG. 8). Since magneto-optic effects benefit from stable, long-lived radicals, QDs are better candidates than their organic counterparts for susceptibility to MOEs. Specifically, they exhibit extended emission lifetimes (on the order of 20 ns), high quantum yield NIR contrast (orders of magnitude greater quantum yield than conventional organic contrast), and tunable absorption wavelengths.

In this family of compounds, one can also modify the quantum dot to tune the wavelength of emission and absorption of the acceptor; other suitable nanoparticles include, for example, semiconductor nanocrystals, metal nanocrystals, hollow nanoparticles, carbon nanospheres, nanorods, nanofibers, nanotubes, nanotori, and the like. In certain embodiments illustrative, the nanoparticle (quantum dot) has a core-shell structure comprising a core comprising a semiconductor material with overcoating. In certain embodiments the overcoating can be a semiconductor material having a composition different from the composition of the core.

1. A contrast marker for use in imaging applications, wherein said marker is sensitive to an applied magnetic field through the formation of radical pair species and wherein said marker is independently magnetically sensitive, optically sensitive, and magneto-optically sensitive.

2. A contrast marker according to claim 1, wherein said marker is a donor-acceptor complex.

3. A contrast agent according to claim 1, wherein said radical pair species include reactive oxygen species, nitroxyl radicals, radical pairs associated with tryptophan and FADH, radical pairs generated from donor-acceptor porphyrin complexes, and radical pairs generated form enzymatic processes.

4. A contrast marker according to claim 1, wherein said contrast marker is sensitive to said magnetic field through the formation of radical pair species that are paramagnetic in the triplet state.

5. A contrast marker according to claim 4, wherein said radical pair species are produced through a charge transfer process.

6. A contrast marker according to claim 2, wherein said marker contains at least two chemical entities covalently linked to form one compound.

7. A contrast marker according to claim 2, wherein the donor or acceptor part of the compound is endogenous to the organism.

8. A contrast marker according to claim 1, wherein said marker is part of a probe, said probe being further comprised of at least one chemical component in order to increase a multimodality thereof.

9. A contrast marker according to claim 8, wherein said multimodality includes electron-spin resonance, magnetic resonance imaging, optical and near-infrared imaging or spectroscopy, x-ray imaging, ultrasound imaging and nuclear imaging techniques.

10. A probe for use in imaging applications, said probe including a contrast marker according to claim 1, said probe being adapted to target a detection and to maximize a change in luminescence response to a magnetic field.

11. A probe for use in imaging applications, said probe including a contrast marker according to claim 1, said probe being adapted to target a detection site and to maximize a change in magnetic response to an optical field.

12. A contrast marker according to claim 1, wherein a luminescence signal produced by said marker under optical, magnetic, or both activation is measured through luminescence intensity, lifetime, spectral properties, or a combination thereof.

13. A contrast marker according to claim 1, wherein a magnetic signal produced by said marker under optical, magnetic, or both activation is measured through a resonance spectral response induced through a radiofrequency pulse.
